(19) United States
(12) Patent Application Publication
(10) Pub. No.: US 2011/0301238 A1
(43) Pub. Date: Dec. 8, 2011

(54) SEIZURE RELATED DISORDERS AND THERAPEUTIC METHODS THEREOF

(76) Inventor: Karin BORGES, TARINGA (AU)

(21) Appl. No.: 13/152,020

(22) Filed: Jun. 2, 2011

Related U.S. Application Data
(60) Provisional application No. 61/350,650, filed on Jun. 2, 2010.

(51) Int. Cl. A61K 31/225; A61P 25/08

(52) U.S. Cl. 514/547

(57) ABSTRACT

Methods of treating seizure disorders by administration of a therapeutically effective amount of at least one precursor of propionyl-CoA in the absence of a ketogenic diet are provided. The present invention particularly applies to administration of triglyceride oils and preferably, triheptanoin and derivatives thereof.

### FIELD OF THE INVENTION

THIS invention relates to the therapy of seizures. More particularly, this invention relates to use of precursors of propionyl-CoA as therapeutic agents for treating a seizure disease inclusive of epilepsy.

### BACKGROUND TO THE INVENTION

Disorders involving seizures can be particularly debilitating. Epilepsy is a neurological disorder that is characterized by unprovoked spontaneously recurring seizures affecting ˜1-3% of the world's population. Currently, there are no anti-epileptogenic drugs that act to prevent, delay, or alter the development of the underlying epileptogenic processes. Up to 30% of epileptic patients, especially children, are drug-resistant and suffer from uncontrolled seizures. Drug-resistant epilepsy is a major public issue that requires more effective treatment options due to the severity of the condition. Also, individuals with drug-resistant epilepsy have 4-7 times increased mortality rate. Lastly, many patients who have reduced seizure severity and/or frequency with antiepileptic drug treatment still suffer from seizures. Evidence is increasing that epileptic disorders are linked to dysfunction of metabolic processes. Several types of epilepsy are caused or linked with metabolic deficiencies, for example glucose transporter 1 deficiency (Klepper and Leiendecker, 2007) and mitochondrial disorders (Kudin et al., 2009, Waldbaum and Patel, 2010). For example, several studies show that manipulation of metabolic pathways and subsequently energy metabolism can be antiepileptic. One of the few alternative and efficacious therapies for drug-resistant epilepsy is the “C4” ketogenic diet (Neal et al., 2008), a strict high fat diet that is very difficult to adhere to and requires discipline. The exact mechanism of seizure control exerted by the C4 ketogenic diet is still unclear, although changes in energy metabolism appear to play a role (Bough et al., 2006, Hartman et al., 2007). Mild hypoglycemia and the replacement glucose by “C4 ketones”, β-hydroxybutyrate and acetoacetate, in energy metabolism may be important for the anticonvulsant effects. Also, it was recently found that fructose-1,6-bisphosphate (Lian et al., 2007) and 2-deoxy-D-glucose (Garriga-Canut et al., 2006, Stafstrom et al., 2009), which both also alter energy metabolism by reducing glycolysis, are effective in several rodent epilepsy models.

### SUMMARY OF THE INVENTION

Despite intensive effort in epilepsy research, there remains a need to treat epilepsy and/or seizures using methods which are comparatively easy to use for a subject.

Therefore in broad forms, the present invention is directed to methods of therapy and compositions comprising anti-convulsants that are useful as an anti-epileptic drug and/or anti-seizure drug.

In another broad form, the invention relates to a method of treating an animal with a seizure disease, disorder or condition, wherein said method includes the step of administering a therapeutically effective amount of at least one precursor of propionyl-CoA to said animal in the absence of a ketogenic diet, to thereby treat said animal with a seizure disease, disorder or condition.

In a first aspect, the invention provides a method of treating an animal with epilepsy and/or other diseases or conditions that cause or result in seizures in an animal, wherein said method includes the step of administering a therapeutically effective amount of at least one precursor of propionyl-CoA to said animal in the absence of a ketogenic diet, to thereby treat said animal with epilepsy and/or other diseases or conditions that cause or result in seizures in said animal.

In a second aspect, the invention provides a pharmaceutical composition for use in the treatment of an animal and/or other diseases or conditions that cause or result in seizures in an animal in the absence of a ketogenic diet, said pharmaceutical composition comprising a therapeutically effective amount of at least one precursor of propionyl-CoA together with a pharmaceutically acceptable carrier, diluent or excipient.

Preferably, the ketogenic diet of any one of the aforementioned aspects is a classical ketogenic diet or a medium chain triglyceride diet, and more preferably, C4 ketogenic diet.

Preferred embodiments relate to the treatment of epilepsy.

Preferably, the at least one precursor of propionyl-CoA is selected from the group consisting of an uneven chain fatty acid, a triglyceride, a C5 ketone body, a phospholipid, a branched amino acid and combinations thereof.

In preferred embodiments, the at least one precursor of propionyl-CoA is a triglyceride or phospholipid of an uneven chain fatty acids.

Preferably, the C5 ketone body is selected from β-hydroxypentanoate and (3-ketopentanoate.

Preferably, the precursor of propionyl-CoA is propionyl-carnitine.

Preferably, the branched amino acid is selected from the group consisting of leucine, valine, isoleucine and combinations thereof.

Even more preferably, the at least one precursor of propionyl-CoA is one or more compounds of Formula I.

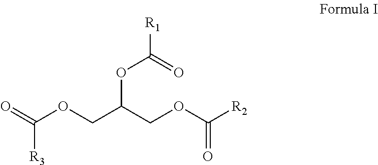

wherein

R₁, R₂ and R₃ are independently selected from alkyl, alkenyl or alkynyl.

Preferably, R₁, R₂ and R₃ are independently selected from C₁ to C₂₀ alkyl, alkenyl or alkynyl.

More preferably, R₁, R₂ and R₃ are independently selected from C₃ to C₁₅ alkyl, alkenyl or alkynyl.

Even more preferably, R₁, R₂ and R₃ are independently selected from C₅ to C₁₂ alkyl, alkenyl or alkynyl.

Still more preferably, R₁, R₂ and R₃ are independently selected from C₆ to C₉ alkyl, alkenyl or alkynyl.

In one preferred embodiment, R₁, R₂ and R₃ are independently selected from methyl, ethyl, propyl, butyl, pentyl, hexyl, heptyl, octyl, nonyl, decyl, undecyl, dodecyl, tridecyl, tetradecyl and pentadecyl, inclusive of all isomers.

Yet even more preferably, the compound of Formula I is triheptanoin.

In other preferred embodiments, the compound of Formula I is trinonanoin.

In yet other preferred embodiments, the compound of Formula I is tripentanoin.

In preferred embodiments, at least one precursor of propionyl-CoA is provided to the animal in an amount comprising at least about 20% of the dietary caloric intake for the animal.

More preferably, at least one precursor of propionyl-CoA is provided to the animal in an amount comprising at least about 25% of the dietary caloric intake for the animal.

Even more preferably, a precursor of propionyl-CoA is provided to the animal in an amount comprising at least about 30% of the dietary caloric intake for the animal.

Yet even more preferably, at least one precursor of propionyl-CoA is provided to the animal in an amount comprising at least about 35% of the dietary caloric intake for the animal.

Preferably, the animal is a mammal.

More preferably, the mammal is a human.

Although the invention is preferably directed to humans, it will be appreciated that the invention is also applicable to other mammals such as livestock, performance animals, domestic pets and the like.

Throughout this specification, unless the context requires otherwise, the words “comprise”, “comprises” and “comprising” will be understood to imply the inclusion of a stated integer or group of integers but not the exclusion of any other integer or group of integers.

### BRIEF DESCRIPTION OF FIGURES AND TABLES

In order that the invention may be readily understood and put into practical effect, preferred embodiments will now be described by way of example with reference to the accompanying:

FIG. 1 The 35% triheptanoin diet is calorically equivalent to the standard diet. A. No significant difference in the growth curves of CF1 mice on standard (black squares) and triheptanoin (white circles) diets for 52 days (n=7; mean+/−SEM). B. Similar daily energy intake per body weight (kcal/g/day) of standard (black bar) and triheptanoin (white bar) diets, showing the diets caloric equivalence. The food intake was averaged for 4 days (n=6 mice per diet group).

FIG. 2 Triheptanoin increased the CC50 in the maximal electroshock threshold test after (A) 3.5 and (B) 6 weeks of treatment (MEST; n=14-15 mice each group, unpaired, two-tailed t-test, A p=0.018, B p<0.0011).

FIG. 3 The development of corneally kindling-induced seizures is delayed by 35% triheptanoin feeding. A, B The median seizure thresholds of each diet group are plotted against the stimulation number, showing significant differences in seizure development with 35% triheptanoin feeding during the kindling process (comparisons of areas under the curve A p<0.05 student's t-test, B P=0.006 One Way ANOVA; P<0.01 35% Triheptanoin vs standard diet, Bonferroni test with multiple comparisons). C: The % of mice with stage 5 seizures are plotted against the stimulation number (same data as B). Black filled squares—standard diet, red triangles—35% triheptanoin, black open squares in B—standard diet w/o sucrose, blue open triangles—20% triheptanoin.

FIG. 4 Lowered PTZ seizure thresholds in the second hit model in the chronic stage of the pilocarpine model correlate with chronic epilepsy. Twenty-three days after pilocarpine injection, SE mice (hatched fill; last bar in series) were more sensitive than no status epilepticus (no SE, white fill; middle bar in series) and “sham control” (diagonal stripe fill; first bar in series) mice to PTZ-induced clonic generalized and tonic seizures (p<0.001, Newman-Keuls post-test after one-way ANOVA with p<0.0001). There were no statistically significant differences between thresholds in no SE and sham control mice (p>0.05).

FIG. 5 Triheptanoin feeding increases the low PTZ tonic extension threshold in the chronic stage of the pilocarpine SE model. A, B Three weeks after pilocarpine injection, SE mice (grey bars) were more sensitive than no SE (white bars) and non-injected control mice to PTZ-induced clonic generalized and tonic seizures (p<0.001, Newman-Keuls post-tests after ANOVA with p<0.0001). B Triheptanoin partially reversed the increased susceptibility to the tonic extension in SE mice (diagonal striped grey bars) p<0.05, Newman-Keuls post-test), but not in no SE mice (diagonal striped white bars).

FIG. 6 CoA profiles. After pilocarpine injection, SE mice and no SE mice were first fed standard diet for two weeks, then divided into groups of equal average weight and received either standard or 35% triheptanoin-containing diet for following three weeks until metabolite quantification. A, B The brain levels of CoA-coupled metabolites are plotted for the different mouse groups in nmol/g wet brain weight. The increase in the levels of the anaplerotic molecules propionyl- and methylmalonyl-CoA found in triheptanoin fed SE mice is consistent with increased anaplerotic flux., white fill; first bar in series—no SE mice standard diet, black fill; third bar in series—SE mice standard diet, diagonal stripe with white background; second bar in series is no SE mice with 35% triheptanoin diet. striped bars with dark fill; fourth and last bar in series—SE mice 35% triheptanoin diet. BHB-β-hydroxybutyrate, HMG-3-hydroxy-3-methylglutaryl, Me-malonyl-methyl-malonyl.

Table 1 Composition of diets. Note that all diets contain the same levels of protein, calcium, magnesium, phosphate, TBHQ, vitamin mix and mineral mix relative to caloric content.

Table 2 Blood acyl-carnitines levels indicate that triheptanoin is metabolised. Mice fed either standard or triheptanoin diet for 3 weeks were sacrificed between 1-4 pm and acyl-carnitines measured in blood by mass spectroscopy (μmoles/L, n=5 mice per diet group). Increases in C7-, C5- and C3 carnitines were significant at the indicated levels (unpaired t-tests) and indicate that triheptanoin is metabolized by mice.

Table 3 Few changes in brain citric acid cycle intermediates and metabolites in SE and no SE mice fed triheptanoin. Two weeks after pilocarpine injection, no Se and SE mice were fed standard or 35% triheptanoin for three weeks. Brain homogenate levels are given in μmol/g wet weight or % of control. One-Way ANOVAs with Tukey post test for each metabolite. Malate, was significantly lower in SE mice relatively to no SE mice on standard diet and BHB levels were doubled by triheptanoin diet. All other steady-state levels of levels of CAC intermediates and products were relatively little affected by SE or diet. BHB-β-hydroxybutyrate.

Table 4 Anticonvulsant properties of triheptanoin compared to current epilepsy treatments in acute and chronic epilepsy models. Efficacy in mouse seizure models is indicated as plus, minus, or some (+/−). Abbreviations: PHB—phenobarbital, PHT—phenyloin, VPA—valproate, CBZ—carbamazepine, LEV—levetiracetam, KD—ketogenic diet, nd—not determined. Note that the PTZ model was performed in rats for the testing of PHB, PHT and VPA. (Klitgaard et al., 1998, Matagne and Klitgaard, 1998, Potschka and Loscher, 1999, Barton et al., 2001, White et al., 2002, Hartman et al., 2008, Blanco et al., 2009, Rowley and White, 2010)

### DETAILED DESCRIPTION OF THE INVENTION

The present invention is predicated, at least in part, on the finding that triheptanoin is anti-epileptic in one acute and two chronic mouse epilepsy models. Administration of triheptanoin increased levels of anaplerotic metabolites in epileptic mouse brains.

In broad aspects, the invention relates to use of precursors of propionyl-CoA for treating and/or ameliorating animals with epilepsy and/or other diseases or conditions that cause or result in seizures in said animal.

In other broad aspects, the invention relates to use of precursors of propionyl-CoA, and more preferably tripheptanoin, as an anti-convulsant in animals, particularly humans, suffering from epilepsy in the absence of a ketogenic diet.

In yet other broad aspects, the invention relates to use of precursors of propionyl-CoA, and more preferably tripheptanoin, as an anti-convulsant in animals, particularly humans, suffering from epilepsy in the absence of a C4 ketogenic diet, which increases the C4 ketones, β-hydroxybutyrate and acetoacetate, but not C5 ketones.

In broad aspects, the invention relates to methods of treating a seizure disease, disorder and/or condition. Seizures are episodes of disturbed brain function that cause changes in attention or behaviour. They are caused by abnormally excited electrical signals in the brain.

A seizure disease, disorder and/or condition may relate to any disease, disorder and/or condition (either neurological or non-neurological) which causes or results in a clinical seizure and includes within its scope epilepsy; gene-related mitochondrial disorders with an epileptic phenotype such as, but not limited to, nuclear-gene related mitochondrial disorders and mitochondrial gene-related mitochondridal disorders (reference is made to Kudin et al, (2009) Experimental Neurology 218: 326-332, which provides non-limiting examples of such mitochondrial disorders with an epileptic phenotype and is incorporated herein by reference), other metabolic disorders such as, hypocalcemia, hypoglycaemia and nonepileptic seizures, hyponatremia, aminoacidurias, hepatic or uremic encephalopathy, hyperglycemia, hypomagnesemia, hypernatremia, vitamin B6 (pyridoxine) deficiency, although without limitation thereto; CNS infections such as but not limited to AIDS, brain abscess, falciparum malaria, meningitis, neurocysticercosis, neurosyphilis, rabies, tetanus, toxoplasmosis, viral encephalitis, drug toxicity or withdrawal; symptomatic seizures that are due to a known cause (eg, brain tumor, stroke); autoimmune diseases such as but not limited to cerebral vasculitis and multiple sclerosis; congenital or developmental abnormalities such as but not limited to, cortical malformations, genetic disorders (eg, fifth day fits, lipid storage diseases such as Tay-Sachs disease), neuronal migration disorders (eg, heterotopias), phenylketonuria; drug and toxin caused seizures including camphor, cocaine and other CNS stimulants, cyclosporine, lead, pentylenetetrazol, picrotoxin, strychnine, tacrolimus; seizure caused by head trauma resulting from birth injury, blunt or penetrating injuries, although without limitation thereto; hyperpyrexia; pressure-related e.g. decompression illness; and withdrawal syndromes.

Suitably, the invention relates to seizure disorders, disorders or conditions wherein the seizures are caused by abnormal electrical discharges or disruptions in the brain such as epilepsy and metabolic orders.

In other suitable embodiments, the invention relates to seizure disorders, disorders or conditions wherein the seizures are behavioural events that are not caused by electrical disruptions of the brain such as non-epileptic seizures. Nonepileptic seizures are classified as having a physiologic or a psychogenic basis.

The preferred methods of the present invention relate to epilepsy or metabolic disorders that cause seizures. In particularly preferred embodiments, the invention relates to epilepsy.

By “epilepsy” (also called epileptic seizure disorder) is meant is a chronic brain disorder characterized by recurrent (≧2), unprovoked seizures. The seizures are caused by sudden, usually brief, excessive electrical discharges in neurons. Epileptic attacks can lead to loss of awareness, loss of consciousness and/or disturbances of movement, autonomic function, sensation (including vision, hearing and taste), mood and/or mental function. Types of seizures include simple partial, complex partial and generalised seizures, such as tonic, clonic, tonic-clonic, absence, Status epilepticus, atonic and myoclonic seizures. The methods of the present invention are particularly suited to treatment of medically refractory epilepsy, chronic epilepsy, acute epilepsy or drug resistant epilepsy.

By “precursor of propionyl-CoA” is meant a substance from which propionyl-CoA can be formed by one or more metabolic reactions taking place within the body.

The invention also includes within its scope the triheptanoin compound itself, as well as salts, prodrugs, analogues, derivatives, substituted, unsaturated, branched forms, or other uneven chain fatty acids and derivatives thereof if applicable.

Typical examples of precursors of propionyl-CoA are uneven-chain fatty acids, in particular seven-carbon fatty acids although without limitation thereto, heptanoate, triglycerides inclusive of triglycerides of an uneven chain fatty acid, a compound of Formula 1, a phospholipid comprising one or two uneven chain fatty acid(s), C5 ketone bodies (e.g. β-ketopentanoate (3-ketovalerate), and β-hydroxypentanoate (3-hydroxyvalerate) but without limitation thereto) (Kinman 2006, Am J Physiol Endocrinol Metab 291 (4): E860-6, Brunengraber and Roe 2006, J Inherit Metabol Dis 29 (2-3): 327-31). The examples of precursors of propionyl-CoA described above include the compounds themselves, as well as their salts, prodrugs, solvates, if applicable.

In particularly preferred embodiments, the at least one precursor of propionyl-CoA is selected from the group consisting of an uneven chain fatty acid, a triglyceride, a phospholipid and combinations thereof.

Preferably, the at least one precursor of propionyl-CoA is an uneven-chain fatty acid and more preferably, a seven-carbon fatty acid. In other preferred embodiments, the at least one precursor of propionyl-CoA is a triglyceride and more preferably a triglyceride of an uneven chain fatty acid. In other preferred embodiments, the at least one precursor of propionyl-CoA is a phospholipid comprising one or two uneven chain fatty acid(s). In other preferred embodiments, the at least one precursor of propionyl-CoA is a C5 ketone bodies. Preferably, the therapeutic agent of the invention is not lacosamide.

Examples of prodrugs include esters, oligomers of hydroxyalkanoate such as oligo(3-hydroxyvalerate) (Seebach 1999, Int J Biol Macromol 25 (1-3): 217-36) and other pharmaceutically acceptable derivatives, which, upon administration to a individual, are capable of providing propionyl-CoA. A solvate refers to a complex formed between a precursor of propionyl-CoA described above and a pharmaceutically acceptable solvent. Examples of pharmaceutically acceptable solvents include water, ethanol, isopropanol, ethyl acetate, acetic acid, and ethanolamine.

In certain preferred embodiments, the precursor of propionyl-CoA is an uneven chain fatty acid. The invention also includes within its scope esters of uneven chain fatty acids. It will be appreciated by a person of skill in the art that an uneven chain fatty acid may also be referred to as an odd-carbon number fatty acid. Preferably, the uneven chain fatty acid is selected from the group consisting of propionic acid, pentanoic acid, heptanoic acid, nonanoic acid and undecanoic acid.

Substituted, unsaturated and/or branched uneven chain fatty acids, as well as other modified uneven chain fatty acids can be used without departing from the scope of the invention.

In other particularly preferred embodiments, the at least one precursor of propionyl-CoA may be one or more compounds of Formula I:

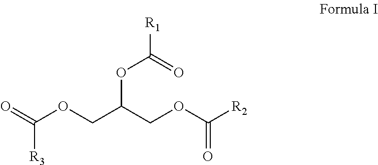

wherein

R₁, R₂ and R₃ are independently selected from alkyl, alkenyl or alkynyl.

Preferably, R₁, R₂ and R₃ are independently selected from C₁ to C₂₀ alkyl, alkenyl or alkynyl.

More preferably, R₁, R₂ and R₃ are independently selected from C₃ to C₁₅ alkyl, alkenyl or alkynyl.

Even more preferably, R₁, R₂ and R₃ are independently selected from C₅ to C₁₂ alkyl, alkenyl or alkynyl.

Still more preferably, R₁, R₂ and R₃ are independently selected from C₅ to C₉ alkyl, alkenyl or alkynyl.

In a particularly preferred embodiment, R1, R2 and R3 are the same and are selected from the group consisting of C₅, C₆, C₇, C₈ and C₉ alkyl, more preferably, selected from C₅, C₇ and C₉ alkyl and yet more preferably, C₇ alkyl.

In one preferred embodiment, R₁, R₂ and R₃ are independently selected from methyl, ethyl, propyl, butyl, pentyl, hexyl, heptyl, octyl, nonyl, decyl, undecyl, dodecyl, tridecyl, tetradecyl and pentadecyl, inclusive of all isomers.

Preferably, R₁, R₂ and R₃ are independently selected from hexyl, heptyl, octyl and nonyl, inclusive of all isomers.

In preferred embodiments the compound of Formula I is an odd-numbered triglyceride. In particularly preferred embodiments, the odd-numbered triglyceride is selected from tripentanoin, triheptanoin and trinonanoin.

In a particularly preferred embodiment, the compound of Formula I is triheptanoin, shown below. This compound may be known by a number of alternative names including 1,3-di(heptanoyloxy)propan-2-yl heptanoate, 1,2,3-propanetriyl triheptanoate and glycerol triheptanoate.

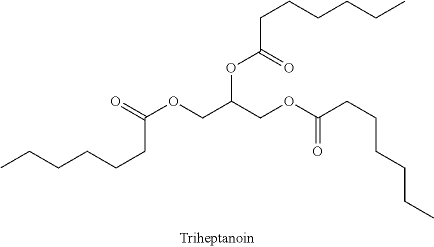

In other preferred embodiments, the compound of Formula I is trinonanoin. This compound may be known by a number of alternative names including glyceroltrinonanoate and glyceryltripelargonate.

The term “alkyl” refers to optionally substituted linear and branched hydrocarbon groups having 1 to 20 carbon atoms. Where appropriate, the alkyl group may have a specified number of carbon atoms, for example, C₁-C₆ alkyl which includes alkyl groups having 1, 2, 3, 4, 5 or 6 carbon atoms in linear or branched arrangements. Non-limiting examples of alkyl groups include methyl, ethyl, propyl, isopropyl, butyl, s- and t-butyl, pentyl, 2-methylbutyl, 3-methylbutyl, hexyl, heptyl, 2-methylpentyl, 3-methylpentyl, 4-methylpentyl, 2-ethylbutyl, 3-ethylbutyl, octyl, nonyl, decyl, undecyl, dodecyl, tridecyl, tetradecyl, pentadecyl.

The term “alkylene” refers to a saturated aliphatic chain substituted at either end, also known as an alkanediyl. Non-limiting examples may include —CH₂—, —CH₂CH₂— and—CH₂CH₂CH₂—.

The term “alkenyl” refers to optionally substituted unsaturated linear or branched hydrocarbon groups, having 2 to 20 carbon atoms and having at least one carbon-carbon double bond. Where appropriate, the alkenyl group may have a specified number of carbon atoms, for example, C₂-C₆ alkenyl which includes alkenyl groups having 2, 3, 4, 5 or 6 carbon atoms in linear or branched arrangements. Non-limiting examples of alkenyl groups include, ethenyl, propenyl, isopropenyl, butenyl, s- and t-butenyl, pentenyl, hexenyl, hept-1,3-diene, hex-1,3-diene, non-1,3,5-triene and the like.

The term “alkynyl” refers to optionally substituted unsaturated linear or branched hydrocarbon groups, having 2 to 20 carbon atoms and having at least one carbon-carbon triple bond. Where appropriate, the alkynyl group may have a specified number of carbon atoms, for example, C₂-C₆ alkynyl groups have 2, 3, 4, 5 or 6 carbon atoms in linear or branched arrangements. Non-limiting examples of alkynyl groups include ethynyl, propynyl, butynyl, penrynyl, hexynyl and the like.

In other preferred embodiments, the invention contemplates administration of a phospholipid comprising one or two uneven chain fatty acid(s). Preferably, the phospholipid is selected from the group consisting of phosphatidic acid (phosphatidate), phosphatidylethanolamine(cephalin), phosphatidylcholine (lecithin), phosphatidylserine and phosphoinositides.

The person skilled in the art is aware of standard methods for production of precursors of propionyl-CoA. A person skilled in the art is be able to determine suitable conditions for obtaining the compounds as described herein, for example, by reference to texts relating to synthetic methodology, non-limiting examples of which are Smith M. B. and March J., March's Advanced Organic Chemistry, Fifth Edition, John Wiley & Sons Inc., 2001 and Larock R. C., Comprehensive Organic Transformations, VCH Publishers Ltd., 1989. Furthermore, selective manipulations of functional groups may require protection of other functional groups. Suitable protecting groups to prevent unwanted side reactions are provided in Green and Wuts, Protective Groups in Organic Synthesis, John Wiley & Sons Inc., 3rd Edition, 1999.

For example, triheptanoin is a triglyceride made by the esterification of three n-heptanoic acid molecules and glycerol and can be obtained by the esterification of heptanoic acid and glycerol by any means known in the art. Triheptanoin is also commercially available through Sasol (Witten, Germany) as Special Oil 107, although without limitation thereto.

Heptanoic acid is found in various fusel oils in appreciable amounts and can be extracted by any means known in the art. It can also be synthesized by oxidation of heptaldehyde with potassium permanganate in dilute sulfuric, acid (Ruhoff, Org Syn Coll. voIII, 315 (1943)). Heptanoic acid is also commercially available through Sigma Chemical Co. (St. Louis, Mo.).

The invention also relates to administration of one or more branched amino acid selected from the group consisting of leucine, valine, isoleucine and combinations thereof.

The amino acids administered in the present invention may be natural or non-natural amino acids, D- or L- amino acids or chemically-derivatized amino acids as are well understood in the art.

It will be appreciated by that a therapeutic effective amount is a sufficient amount of at least one precursor of propionyl-CoA to substantially alleviate, ameloriate, reduce and/or eliminate one or more symptoms of a seizure disease, disorder or conditions. In embodiments that relate to epilepsy, this is inclusive of seizures, depression and/or mental impairment.

The present invention includes within its scope a therapeutic amount of at least one precursor of propionyl-CoA is less than 100% of dietary caloric intake and preferably, within a range from between about 5% and about 90%, more preferably between about 15% and about 80%, even more preferably between about 20% and about 60%, yet even more preferably between about 25% and 50% and more preferably between about 30% and about 40%.

In particularly preferred embodiments, at least one precursor of propionyl-CoA is provided to the animal in an amount comprising at least about 5%, at least about 10%, at least about 15%, at least about 20%, at least about 20.5%, at least about 21%, at least about 21.5%, at least about 22%, at least about 22.5%, at least about 23%, at least about 23.5%, at least about 24%, at least about 24.5% at least about 25%, at least about 25.5%, at least about 26%, at least about 26.5%, at least about 27%, at least about 27.5%, at least about 28%, at least about 28.5%, at least about 29%, at least about 29.5%, at least about 30%, at least about 30.5%, at least about 31%, at least about 31.5%, at least about 32%, at least about 32.5%, at least about 33%, at least about 33.5%, at least about 34%, at least about 34.5%, at least about 35%, at least about 35.5%, at least about 36%, at least about 36.5%, at least about 37%, at least about 37.5%, at least about 38%, at least about 38.5%, at least about 39%, at least about 39.5%, at least about 40%, at least about 40.5%, at least about 41%, at least about 41.5%, at least about 42%, at least about 42.5%, at least about 43%, at least about 43.5%, at least about 44%, at least about 44.5%, at least about 45%, at least about 45.5%, at least about 46%, at least about 46.5%, at least about 47%, at least about 47.5%, at least about 48%, at least about 48.5%, at least about 49%, at least about 49.5%, at least about 50%, at least about 55%, at least about 60%, about at least about 70%, at least about 80%, at least about 90% or more of the dietary caloric intake.

It will be appreciated by a skilled addressee that “% of dietary caloric intake” may relate to % of kJoules or % of kcal.

By “ketogenic diet” is meant a high fat and low carbohydrate and protein diet. Typically, a ketogenic diet contains a 3:1 to 4:1 ratio by weight of fat to combined protein and carbohydrate. A ketogenic diet may refer to a classical ketogenic diet comprising predominantly natural fats (inclusive of normal dietary fats and suitably long-chain triglycerides) or a ketogenic diet comprising predominantly medium chain triglycerides and suitably, even medium chain triglycerides.

In the context of the present invention, by “absence of a ketogenic diet” is meant a dietary intake which does not have a higher than normal fat content compared to carbohydrate and protein. In some preferred embodiments, an “absence of a ketogenic diet” is a diet is which the ratio by weight of fat to combined protein and carbohydrate is less than 3:1, and may be 2:1, 1:1, 0.5:1 or a ratio where the fat content is even lower, or where fat is absent. The ketogenic diet may be a classical ketogenic diet or a medium chain triglyceride ketogenic diet as hereinbefore described.

By “C4 ketogenic diet” is meant a high fat and low carbohydrate and protein diet. Typically, a C4 ketogenic diet contains a 3:1 to 4:1 ratio by weight of even chain fat to combined protein and carbohydrate. A C4 ketogenic diet increases the C4 ketones, β-hydroxybutyrate and acetoacetate, but not C5 ketones. A C4 ketogenic diet may refer to a classical ketogenic diet comprising predominantly natural fats (inclusive of normal dietary fats and suitably long-chain triglycerides) or a ketogenic diet comprising predominantly medium chain triglycerides and suitably, even medium chain triglycerides, mostly C8 and C10 oil.

In the context of the present invention, by “absence of a C4 ketogenic diet” is meant a dietary intake which does not have a higher than normal even chain fat content compared to carbohydrate and protein. In some preferred embodiments, an “absence of a C4 ketogenic diet” is a diet is which the ratio by weight of fat to combined protein and carbohydrate is less than 3:1, and may be 2:1, 1:1, 0.5:1 or a ratio where the fat content is even lower, or where fat is absent. These embodiments may relate to the absence of a classical C4 ketogenic diet or the absence of a medium chain triglyceride C4 ketogenic diet.

In preferred embodiments, the invention relates to methods of therapy in the absence of a substantially ketogenic diet (as described hereinbefore) and preferably, in the absence of a substantially C4 ketogenic diet (as described hereinbefore).

The invention is particularly suited to the treatment of adult animals but the invention does contemplate treatment of juvenile animals.

Typically, pharmaceutical compositions according to the invention comprise at least one precursor of propionyl-CoA together with a pharmaceutically-acceptable carrier, diluent or excipient. In particularly forms, the pharmaceutical composition is dietary formulation or a nutritional supplement and it will be that according to these embodiments, the therapeutic agent may be food-grade or be a constituent of a formulation which is food grade.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

A useful reference describing pharmaceutically acceptable carriers, diluents and excipients is Remington's Pharmaceutical Sciences (Mack Publishing Co. N.J. USA, 1991) which is incorporated herein by reference.

Any safe route of administration may be employed for providing a patient with the at least one precursor of propionyl-CoA containing composition of the invention. For example, enteral, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed. Preferably, it can be administered via ingestion of a food substance containing triheptanoin at a concentration effective to achieve therapeutic levels. Alternatively, it can be administered as a capsule or entrapped in liposomes, in solution or suspension, alone or in combination with other nutrients, additional sweetening and/or flavoring agents. Capsules and tablets can be coated with shellac and other enteric agents as is known.

Dosage forms include tablets, dispersions, suspensions, injections, solutions, syrups, oils troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Compositions of the present invention suitable for enteral, intraperitoneal, oral or parenteral administration may be presented as discrete units such as capsules, sachets or tablets each containing a pre-determined amount of the therapeutic agent of the invention, as a powder or granules or as a solution or a suspension in an aqueous liquid, a non-aqueous liquid, an oil-in-water emulsion or a water-in-oil liquid emulsion. Preferably, administration of the agent of the invention is by way of oral administration. Such compositions may be prepared by any of the methods of pharmacy but all methods include the step of bringing into association one or more agents as described above with the carrier which constitutes one or more necessary ingredients. In general, the compositions are prepared by uniformly and intimately admixing the agents of the invention with liquid carriers or finely divided solid carriers or both, and then, if necessary, shaping the product into the desired presentation.

The above compositions may be administered in a manner compatible with the dosage formulation, and in such amount as is pharmaceutically-effective. The dose administered to a patient, in the context of the present invention, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner.

It will also be appreciated that treatment methods and pharmaceutical compositions may be applicable to prophylactic or therapeutic treatment of mammals, inclusive of humans and non-human mammals such as livestock (e.g. horses, cattle and sheep), companion animals (e.g. dogs and cats), laboratory animals (e.g. mice rats and guinea pigs) and performance animals (e.g racehorses, greyhounds and camels), although without limitation thereto.

So that the invention may be readily understood and put into practical effect, the following non-limiting Examples are provided.

### EXAMPLES

### Example 1

### Antiepileptic Effects of a Triheptanoin Diet in Two Chronic Mouse Epilepsy Models

### Materials and Methods

### Diets and Mice

All mice were housed under a 12 h light dark cycle with free access to food and water. Adult male CF1 mice (20-43 g, Charles River, USA) were fed either a standard diet (TD.06316, as used by (Samala et al., 2008)), standard diet without sucrose, or diet containing either 20% or 35% of calories from triheptanoin (Sasol, Germany, table 1). Adult male CD1 mice (20-35 g, Australian Institute for Bioengineering and Nanotechnology, Australia) were fed similar diet, prepared by Western Specialty Feed (West Australia). Triheptanoin replaced sucrose and some of the complex carbohydrates in the standard diet. The amounts of vitamins, minerals, antioxidants and protein match the newest nutritional standards and were equal among all diets relative to their caloric densities. Diets were mixed fresh every 3-4 days in the laboratory, dried and then supplied to the mice. Mice were initially weighed daily to ensure adequate nutrition.

### Caloric Intake and Metabolism Studies

To compare caloric intake in mice fed standard vs. 35% triheptanoin diet, twelve 28-34 g mice were placed individually in metabolic chambers and fed respective diets for eight days. After 4 days habituation, food intake and body weight were determined daily and then averaged per day for each mouse. 24 h urine samples were taken and organic acids isolated by liquid partition chromatography and trimethylsilyl derivatives quantified by gas chromatography mass spectrometry (Sweetman, 1991). In a separate study mice were fed respective diets for 3 weeks and were decapitated after isoflurane anaesthesia between 1-4 pm. Trunk blood was collected on absorbent filter paper for measurement of acyl-carnitine levels in dried blood spots using tandem mass spectrometry modified from the method of Rashed et al. (Rashed et al., 1997).

### Maximal Electroshock Threshold Test

0.5% Tetracaine (Sigma Aldrich) in 0.9% NaCl solution was applied to corneas 30 minutes prior to stimulation. Saline solution was also applied to corneas and electrodes immediately prior to stimulation to ensure solid conduction. Mice were administered corneal electroshocks, which were generated using a Hugo Sachs Type 211 rodent shocker (Harvard Apparatus, Germany). Stimulation parameters of 50-60 Hz sine wave frequency, 0.2 second shock duration starting at an 11 mA current were used (Giardina and Gasior, 2009). Immediately following shock administration mouse seizure behaviour was observed. Severe clonic convulsions followed by tonic extension of both forelimbs were classified as seizures. 1 mA increments were used to determine the seizure threshold CC50 value using the up down method (Kimball et al., 1957). All mice were terminated via cervical dislocation immediately following seizure observational time.

### Corneal Kindling

The corneal kindling model was slightly modified from the original protocol (Matagne and Klitgaard, 1998) with kindling twice a day with at least 4 h intervals. A topical anesthetic (0.5% tetracaine hydrochloride ophthalmic solution) was applied to the corneas 10-15 min before stimulation. After wetting the electrodes with 0.9% NaCl immediately before testing, 9 mA 0.4 ms duration pulses at 50 Hz were applied to the corneas for 3 seconds by a constant-current device (ECT Unit 57800, Ugo Basile). Mice were manually held during stimulation and then released for behavioral observation. Seizures were scored according to a modified Racine scale (Racine, 1972) as described by Matagne and Klitgaard (1998) with 0=no reaction or immobility; 1=jaw clonus; 2=myoclonic twitches in the forelimbs, sometimes with head nodding; 3=clonic convulsions in the forelimbs; 4=clonic convulsions in the forelimbs with rearing and falling; 5=loss of balance. To investigate if triheptanoin is anti-convulsant in the fully kindled stage, mice on standard diet were kindled for two weeks until at least four stage 5 seizures were obtained. Mice were then randomized and placed on 35% triheptanoin vs. standard diet. Both groups had the same average seizure scores for the full kindling period and the last three days of kindling. During the five following weeks, mice were rekindled every week twice a day on two consecutive days and the scores were averaged.

Pilocarpine Model with Second Hit Seizure Models

The pilocarpine CF1 mouse model used in this study was slightly modified from our previous description (Borges et al., 2003, Borges et al., 2004, Borges et al., 2006, Borges et al., 2008). 26-43 g CF1 mice were injected with methylatropine (2 mg/kg i.p. in 0.9% NaCl) to minimize peripheral side effects followed after 15-30 min by different doses of pilocarpine for CF1 mice (270-360 mg/kg, s.c.). About fifty percent of injected mice experienced behavioral status epilepticus (SE) lasting about four hours as defined by continuous seizure activity consisting mainly of whole body continuous clonic seizures. 4.5 h after pilocarpine injection, all mice were injected with pentobarbital (22.5 mg/kg, i.p.) followed by 1 ml 5% dextrose in lactate Ringer's solution (i.p.). After SE, mice were monitored daily, hand-fed moistened cookies and injected with 5% dextrose in lactate Ringer's solution twice a day for about three days and thereafter when needed. Spontaneous and handling-induced seizures, including stage 3-5 seizures, jumping and wild running, were noted. When video-monitored all mice with SE developed spontaneous recurrent seizures (Borges et al., 2003), while those that do not develop SE (no SE mice) have never been observed to have handling-induced or spontaneous seizures or neuronal damage in our laboratory. “Sham control” mice received methylatropine and pentobarbital, but saline instead of pilocarpine. Out of a total of 205 mice injected with pilocarpine, the inventors obtained 44% SE, 41% no SE mice, while 14% of mice died.

In the chronic stage of the pilocarpine model, the seizure thresholds for the first generalized clonic and tonic seizures induced by pentylenetetrazole (PTZ, i.v.) as a measure for seizure susceptibility of SE, no SE and control mice were assessed. As previously described (Samala et al., 2008, Willis et al., 2009), 10 mg/ml PTZ dissolved in saline was infused into the tail vein at 150 μl/min. The latencies to the first generalized myoclonic seizure and tonic extension were determined and converted to seizure thresholds expressed as mg/kg body weight. Mice were euthanized by cervical dislocation immediately after the tonic extension seizure.

### Brain and Blood Metabolite Analysis

To determine to which extent the 35% triheptanoin diet affects brain metabolite levels in a chronic epilepsy model, the brain metabolite profile in the chronic stage of the pilocarpine model in SE mice to “non-epileptic” no SE mice were compared. Both groups of mice received standard diet during the phase of epileptogenesis, the first two weeks after SE. They were then divided into two groups of equal average weight and received either standard or 35% triheptanoin-containing diet for three weeks. To avoid metabolite changes induced by anesthesia, mice were killed by cervical dislocation and then decapitated. Within 22-32 seconds brains without cerebellum were frozen in liquid nitrogen. Trunk blood was collected in heparinized tubes for analysis of β-hydroxybutyrate and glucose using kits from Pointe Scientific and Raichem, respectively. Brains were stored at −80 C and shipped to the Metabolic Mouse Phenotyping Center at Case Western University for chromatography mass spectrometry (GC-MS/LC-MS) analysis and characterization of intermediates and products of the CAC and acyl-CoA's.

### Statistics

All data points are presented as averages±standard error of the mean (s.e.m.). Unpaired two-tailed t-tests were used to compare body caloric intake and blood metabolite levels in two groups. Repeated measure ANOVAs with a subsequent post-test were employed to compare body weights in mice on different diets. To compare kindling scores over time, the areas under the curve for each mouse and compared them with unpaired two-tailed student's t-test for two diet groups were calculated. One way ANOVAs followed by the Newman-Keuls test were used to compare the areas under the curves between different diet groups and seizure thresholds in the PTZ test. To compare levels of individual metabolites after SE and with the triheptanoin diet, one way ANOVAs with a Bonferroni test with selected comparisons was used. GraphPad Prism version 5 was used for all statistical tests. Significance was set to p<0.05. * depicts p<0.05, **p<0.01, and ***p<0.001.

### Results

### Diets, Body Weights and Triheptanoin Metabolism

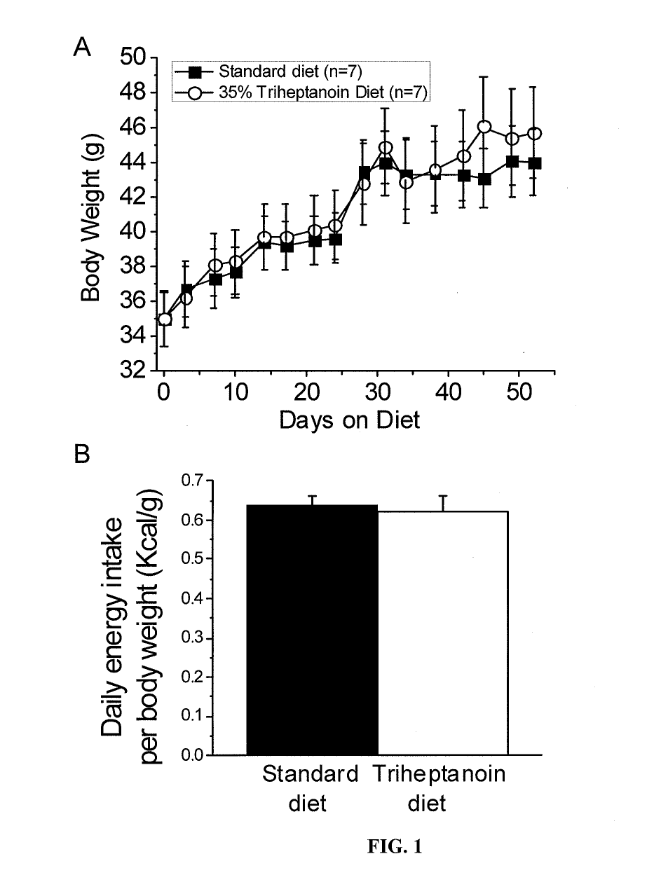

CF1 mice were fed 35% triheptanoin diet for up to 52 days. Their visual appearance was indistinguishable from mice fed standard chow and body weights were statistically indistinguishable (FIG. 1A, repeated measures ANOVA). Caloric intake measured in metabolic cages was also similar among triheptanoin and standard diet-fed mice, indicating that triheptanoin is well tolerated and metabolized in mice (FIG. 1B).

To ensure that triheptanoin is metabolized as expected by fatty acid oxidation acyl-carnitine levels in blood were measured. Intracellular acyl-carnitines are in equilibrium with fatty acid acyl-Coenzyme A intermediates in mitochondrial fatty acid beta oxidation was measured. The acyl-carnitines can enter the blood where their levels reflect the intracellular levels of fatty acid acyl-Coenzyme As and can be used as an indicator of fatty acid metabolism. In triheptanoin-fed mice, heptanoyl-, pentanoyl- and propionyl-carnitines were elevated significantly two -3.4-fold in the afternoon (p=0.002-0.012, n=5, table 1), without any differences in even chain fatty acid carnitine metabolites, indicating that triheptanoin is metabolized to C7-, C5- and C3-fatty acid metabolites. Please note that C5- and C7-carnitines are more substantially elevated shortly after feeding (data not shown). Analysis of organic acids in urine showed some increases in markers for propionic acidemia, such as methylmalonate and methylcitrate. These levels were well below those found in patients suffering from propionic acidemia, indicating that triheptanoin feeding does not lead to pathological propionic acid overload.

### Effect of the Triheptanoin Diet on Seizure Susceptibility

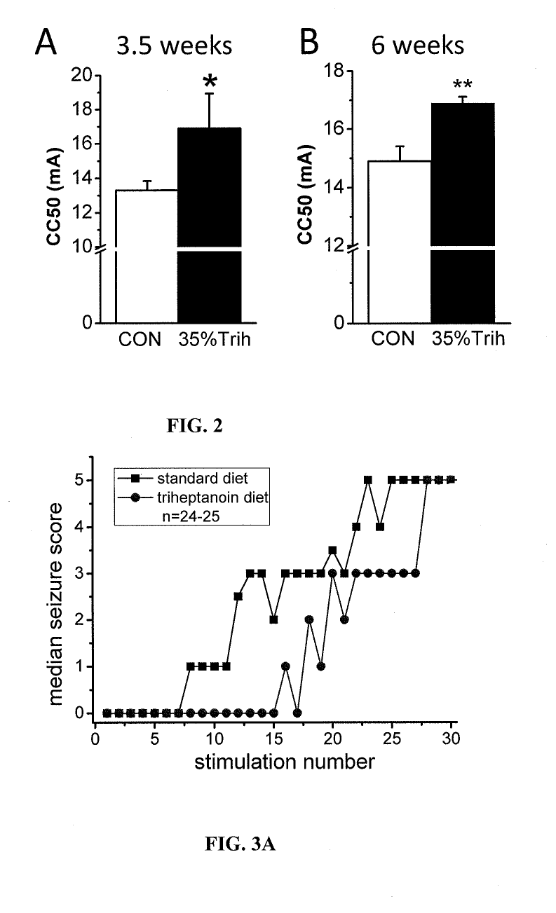

After 3.5 and six weeks of feeding 35% of daily caloric intake as triheptanoin oil we observed a reproducible increase of the MEST (the critical current at which 50% of mice seize) in the acute maximal electroshock threshold test (MEST), a test for the efficacy against generalised seizures, in CD1 mice (FIG. 2A p=0.018, B p<0.0011).

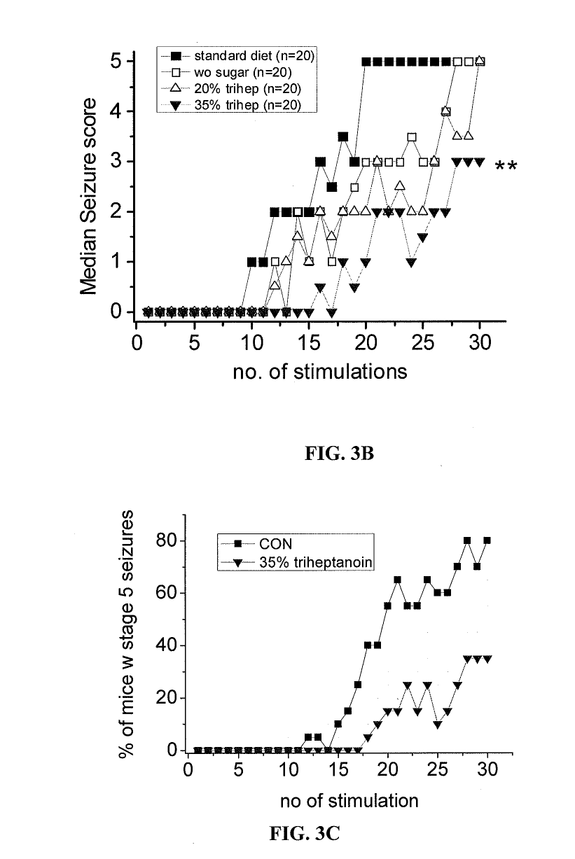

The effect of 35% triheptanoin feeding on corneal kindling was investigated in two independent experiments in CF1 mice, with triheptanoin feeding initiated three weeks prior to and continued throughout the kindling process. This feeding regimen delayed the development of kindled seizures (FIG. 3A p<0.05, t-test; B p<0.01 one way ANOVA and subsequent Newman-Keuls test, C same data as B, plotted in a different way). In the second experiment, the inventors also evaluated if the omission of sucrose in the triheptanoin diet accounts for the antiepileptic effect and if 20% triheptanoin is sufficient to affect kindling. FIGS. 3B and 3C show that the kindling development was not statistically different with omission of sucrose or 20% triheptanoin relative to the standard diet, indicating that more than 20% of triheptanoin is required in a rodent model.

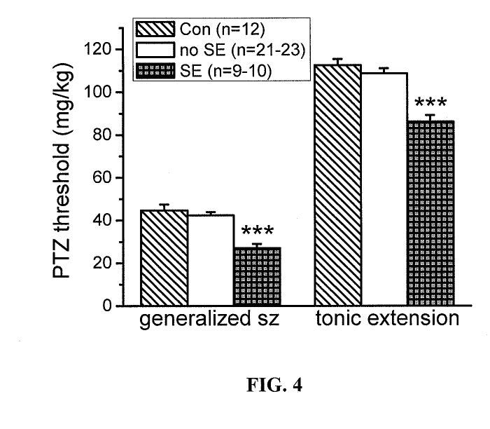

To confirm the anti-epileptic effect found in the corneal kindling model, another chronic epilepsy model was developed in CF1 mice. Video-EEG analysis of spontaneous seizures in chronic epilepsy models is a complicated and time-consuming procedure. The inventors therefore established a second hit model, in which mice in the chronic stage of the pilocarpine model show increased seizure sensitivities to induced seizures. Fourteen to 24 days after pilocarpine-induced SE, mice with SE were significantly more sensitive to PTZ-induced seizures (FIG. 4, p<0.001, n=4 experiments). “Sham control” and no SE mice showed no statistically significant difference in their thresholds to the PTZ-induced first generalized and tonic extension seizures (p>0.05 Newman-Keuls post-test after ANOVA), justifying the use of no SE mice as control mice (FIG. 4).

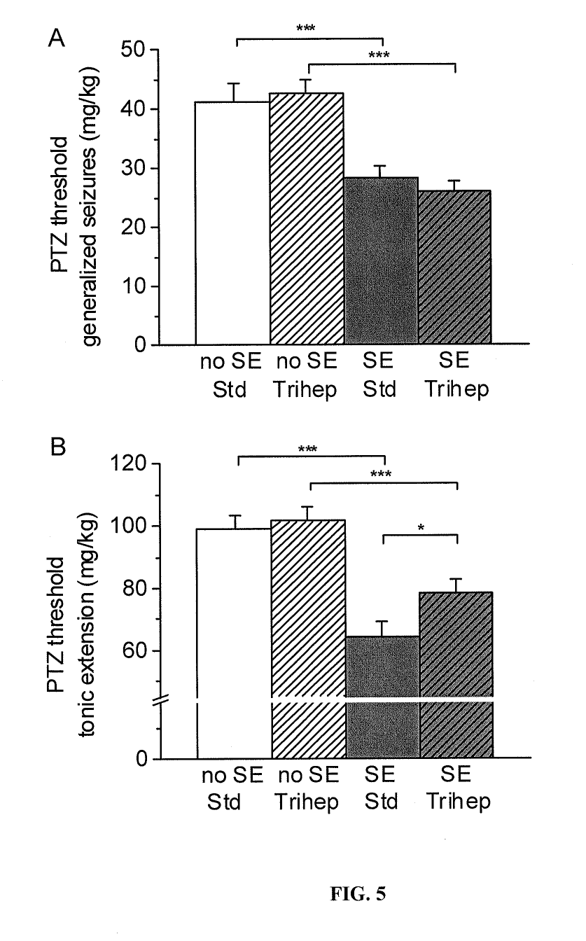

Two experiments were performed to determine the effect of triheptanoin after the epileptogenic insult of SE. First, triheptanoin or standard diets were initiated immediately after pilocarpine injection to SE, no SE and control mice for three weeks until the PTZ threshold test (FIG. 5). The PTZ seizure thresholds to generalized clonic seizures and tonic extension were 30-35% lower in SE mice compared to the combined no SE and control mouse group (P<0.0001 ANOVA, p<0.001 post-hoc Newman-Keuls test, n=10-16 mice). Triheptanoin reversed this increased susceptibility to the tonic extension by about 40% in the SE mice (p<0.05 Newman-Keuls test), indicating that triheptanoin is anti-epileptic and may prevent seizure spread in mice with chronic epilepsy. Triheptanoin had no effect on PTZ clonic seizure thresholds in SE or no SE mice. Spontaneous and handling-induced seizures were observed in SE mice on either diet to a similar extent, i.e. six out of 16 SE mice on standard diet and eight out of 17 SE mice on triheptanoin diet (p=0.73, Fisher's exact test), showing that triheptanoin does not prevent epileptogenesis after pilocarpine-induced SE.

In the second experiment, three week triheptanoin feeding was initiated in the chronic stage of the model at two weeks after pilocarpine injection (the same time course as the metabolite analysis, see below). Again, triheptanoin counteracted a 30% increased susceptibility to tonic extension seizure in SE mice by about 50% (P<0.0001 ANOVA, p<0.05 post hoc Newman-Keuls test, n=14-16 mice). This finding indicates that triheptanoin may also be anti-epileptic when administered after epilepsy has developed.

### Blood and Brain Metabolites in the Chronic Stage of the Pilocarpine Model

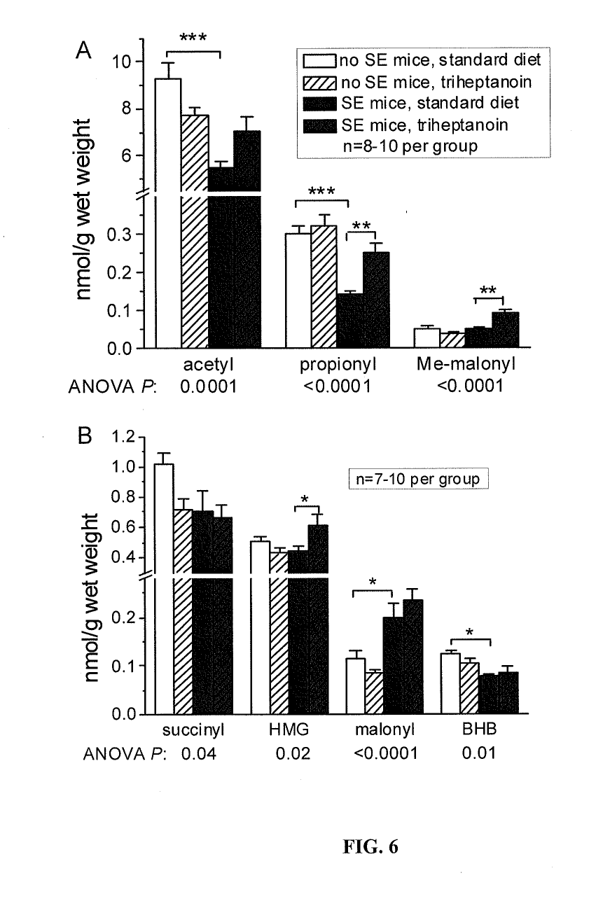

To investigate to which extent triheptanoin feeding induced changes in brain metabolism that could account for the anti-epileptic effects found, the inventors compared brain metabolite levels in the chronic stage of the pilocarpine model in SE relative to “non-epileptic” no SE CF1 mice (FIG. 6, table 3). Two weeks after pilocarpine injection, mice were placed on triheptanoin vs. standard diet for three weeks. During the five weeks after SE all SE mice, except for one mouse on the standard diet, were observed with either spontaneous or handling-induced behavioral seizures. When on standard diet, levels of certain metabolites were statistically significantly different in SE compared to no SE mice, including a 1.8-fold increase in malonyl-CoA concentrations and decreases in the levels of propionyl- (50% loss), acetyl- and βhydroxybutyryl-CoA (both 40%, all ANOVAs p≦0.01 and post-hoc Bonferroni tests with selected comparisons P<0.05; FIG. 6). Also, in SE mice the levels of aspartate (29%), malate (23%) and γ-aminobutyric acid (GABA, 15%) were decreased with statistical significance of <0.05 (post-hoc Bonferroni test with selected comparisons; all ANOVAs p≦0.01, table 3), supporting our hypothesis that epileptic tissue shows changes in metabolism, potentially including CAC (Citric acid cycle) activity. Triheptanoin feeding to SE mice restored only the brain propionyl-CoA levels (FIG. 5, ANOVA p<0.001, Bonferroni test with selected comparisons p<0.001) and largely increased the levels of methylmalonyl- (1.4-fold) and HMG-CoA (1.8-fold) in chronically epileptic SE mice, but not in no SE mice. On the other hand, triheptanoin feeding to no SE mice increased malate (26%) and decreased fumarate (17%) levels (p<0.05, both Bonferroni post-hoc tests with selected comparisons), suggesting a potential increase in fumarase activity. Last, the 35% triheptanoin diet doubled the brain β-hydroxybutyrate levels in both SE and no SE brains (table 3) which may be explained by the high dietary fat content in the absence of sucrose. On the other hand, there were no significant changes in plasma levels of β-hydroxybutyrate (0.17-0.28 mM) or glucose (174-228 mg/dl). No significant changes by SE or diet were found in the steady state brain levels of the CAC intermediates succinyl-CoA, succinate and citrate and its metabolites glutamate, and glutamine(table 3). In summary, these measurements of steady state metabolite levels do not show any clear evidence of anaplerosis by triheptanoin in the “non-epileptic” or “epileptic” brain.

### Discussion

Our principal findings on the effects of triheptanoin in acute and chronic mouse epilepsy models are: 1) Our 35% triheptanoin diet is calorically equivalent to a rodent standard diet and is well tolerated by outbred CF1 mice over up to 7.5 weeks of feeding. 2) 35% triheptanoin was repeatedly antiepileptic in one acute MEST model and two chronic mouse epilepsy models, during the development of corneal kindling and the PTZ threshold test in mice in the chronic stage of the pilocarpine model. The omission of sucrose in the diet does not account for this effect and 20% triheptanoin was not sufficient to delay corneal kindling. 3) Mice in the chronic stage of the pilocarpine model showed changes in steady state brain metabolite levels, some which were restored by triheptanoin feeding. Taken together these findings indicate that triheptanoin is antiepileptic.

Anaplerotic molecules include amino acids and odd chain fatty acids. Triheptanoin is the triglyceride of the anaplerotic fatty acid heptanoate and provides three anaplerotic propionyl-CoA molecules without overloading the system with nitrogen, sodium or acid. Heptanoate itself can enter the brain (Wang et al., 2007). Moreover, the liver metabolizes heptanoate to the “C5 ketone” bodies hydroxypentanoate & β-ketopentanoate, which are taken up by the brain, most likely through the monocarboxylate transporters. Each C5 ketone molecule is metabolized to one acetyl-CoA and one anaplerotic propionyl-CoA molecule. Alternatively, propionyl-CoA can be produced by β-oxidation of heptanoyl-CoA. Propionyl-CoA can replenish oxaloacetate via succinyl-CoA in rats and thus can increase acetyl-CoA oxidation and ATP production in humans and rodents (Kinman et al., 2006).

### Anti-Epileptic Profile of Triheptanoin

Triheptanoin feeding was effective at 35% of the caloric intake in one acute and two chronic seizure models, the second hit PTZ model in pilocarpine-SE mice and corneal kindling. Elevation of the CC50 in the MEST model may indicate efficacy of triheptanoin against generalized seizures, as found with many other antiepileptic drugs, e.g. phenobarbital, phenyloin, valproate and carbamazepine (see table 4). No consistent anticovulsant effect was found with 30% triheptanoin in acute seizure models, including the PTZ (i.v.), fluorothyl and 6 Hz models (Willis and Borges in preparation). Efficacy in certain seizure models has been correlated to efficacy in different human seizure types, e.g. (White, 2003, Smith et al., 2007). Triheptanoin's anticonvulsant profile is unusual. To our knowledge, only a few antiepileptic drugs have been previously tested during corneal kindling development, namely brivaracetam and leviteracetam (Matagne et al., 2008). Triheptanoin delays kindling in a similar fashion as those two drugs at low concentrations. Also, the delay of kindling by triheptanoin in the corneal kindling model mirrors the effect of valproate, phenobarbital and lacosamide during amygdala kindling in the rat (Silver et al., 1991, Brandt et al., 2006), indicating that triheptanoin may be a powerful clinically effective treatment for various seizure types.

Our second hit model is a new model in the mouse, but a similar second hit model was recently developed in the rat (Blanco et al., 2009). Blanco and colleagues treated Wistar rats with pilocarpine and one month later SE and no SE rats were subjected to the subcutaneous PTZ test. The PTZ-induced seizures were sensitive to valproate, phenobarbital and phenyloin in no SE rats, but not in SE rats. These data suggest that the PTZ model in animals with SE may be a useful tool to find treatments with efficacy in pharmacoresistant epilepsy. Given that rats and mice show similarities in many seizure models, including their response to different anticonvulsant drugs in the PTZ model (Loscher et al., 1991) and the pathophysiological changes after pilocarpine-induced SE (e.g. (Turski et al., 1983, Turski et al., 1984, Borges et al., 2003, Curia et al., 2008), it is likely that our mouse model is also fairly pharmacoresistant.

### Metabolite Changes

The metabolites levels measured are within range of published levels in rodents (e.g. (Goldberg et al., 1966, Nordstrom et al., 1978, Bough et al., 2006, Puchowicz et al., 2008). The levels vary to some degree, which can be explained by different brain extraction methods (Goldberg et al., 1966). Epileptic tissue showed changes in metabolite levels, some of which may contribute to seizures. There are several reasons why a therapeutic approach to increase anaplerosis in the brain appears to be viable. It is plausible that CAC cycle intermediates are reduced in chronic epilepsy, because α-ketoglutarate is the precursor for the neurotransmitters glutamate and GABA and oxaloacetate for aspartate. Increased neurotransmission, that is, release of these substances, such as during seizures, can reduce the levels of CAC cycle intermediates. The following studies in three different epilepsy models during the chronic phase corroborate this notion. In this example, we found that in PILO-SE mice forebrain levels of malate and also propionyl-CoA were decreased. In the hippocampal formation of lithium PILO-SE rats levels of glutamate, aspartate (indicative of the TCA intermediates α-ketoglutarate and oxaloacetate), N-acetyl aspartate, adenosine triphosphate plus adenosine diphosphate and glutathione were decreased (Melo et al., 2005) Glutamate concentrations were also decreased in the hippocampi of rats after kainate-induced SE (Alvestad et al., 2008). These findings are consistent with our hypothesis that increased anaplerosis might protect “epileptic” brains against seizures.

The increase in the levels of the anaplerotic molecules propionyl- and methylmalonyl-CoA found in triheptanoin fed SE mice is consistent with increased anaplerotic flux. The anaplerotic need of the CAC is expected to be increased in epileptic tissue, because CAC intermediates are the precursors of neurotransmitters, such as glutamate and GABA, which are excessively released during seizures. If this need is not fully met, energy shortage ensues, which could lead to depolarization of neuronal membranes and lower seizure threshold. The inventor hypothesized that triheptanoin feeding would be anaplerotic in the epileptic brain, which could provide additional ATP and potentially GABA and may therefore protect against seizure generation. While it was found that triheptanoin was antiepileptic, it is yet unclear to which extent triheptanoin feeding was anaplerotic in the brain. Our analysis of steady state brain metabolite levels revealed that triheptanoin feeding increased levels of anaplerotic precursor molecules in chronically epileptic brains. However, there were no significant changes in the steady state levels of the CAC intermediates or metabolites quantified in either the SE or no SE mice that point to anaplerosis. On the other hand, the turnover of the CAC is fast and small undetectable changes in steady state metabolite levels may still largely increase the capacity to produce ATP. Future work using ¹³C tracers and mass isotopomer analysis is needed to determine the degree of anaplerotic fluxes and to understand the metabolic fates of propionyl-CoA.

### Conclusion

The inventors have developed a new anti-epileptic palatable diet for rodents, which is tolerated well by mice. The inventors have demonstrated that the triheptanoin diet is anti-epileptic in one acute and two chronic mouse epilepsy models. In the chronic epilepsy stage in pilocarpine-SE mice, a significant reduction in brain propionyl-CoA levels was revealed, which was largely restored by triheptanoin feeding. Whether these metabolic changes underlie triheptanoin's anti-epileptic effect remains to be studied. This work provides the foundation to future studies aimed at optimizing this diet for the treatment of human epilepsy and the deciphering its antiepileptic mechanism.

### Example 2

Table 4 summarises and compares the anticonvulsant profiles of triheptanoin, common antiepileptic drugs and C4 ketogenic therapies. In summary, triheptanoin's anticonvulsant profile is unique. Also, as discussed above triheptanoin shows unique efficacy in models that appear to similar to drug-resistant epilepsy, e.g. the PTZ second hit pilocarpine model and the 6 Hz model, indicating that it is potentially effective in patients with medically refractory epilepsy.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein is incorporated herein by reference.

### TABLES

TABLE 1
Composition of diets

| | Standard | |
|---|---|---|
| | (TD06316) | 35% Triheptanoin diet |
| | g/kg | g/kg |
| Casein | 200.0 | 215.0 |
| Corn Starch | 389.1 | 350.2 |
| Maltodextrin (Lo-Dex) | 100.0 | 89.8 |
| Sucrose | 150.0 | |
| Cellulose | 50.0 | 102.6 |
| Mineral Mix, Ca—P Deficient | 13.4 | 14.3 |
| (79055) | | |
| Vitamin Mix, Teklad 40060 | 10.0 | 10.7 |
| Calcium Phosphate Dibasic | 7.5 | 8.0 |
| Calcium Carbonate | 6.85 | 7.40 |
| DL-Methionine | 3.00 | 3.24 |
| Magnesium Oxide | 0.20 | 0.22 |
| TBHQ (Antioxidant) | 0.07 | 0.08 |
| Choline Bitartrate | | 2.50 |
| Pantothenic acid | | 1.93 |
| Vegetable Oil, Hydrogenated | 50.0 | |
| (Crisco) | | |
| Coconut Oil, Hydrogenated | 20.0 | |
| Triheptanoin | | 170.5 |
| Corn oil | | 23.5 |
| total (g) | 1000 | 1000 |
| Kcal/g | 3.77 | 4.01 |
| % kcal/kcal diet: | | |
| Protein | 18.9 | 18.9 |
| Carbohydrates | 63.7 | 40.2 |
| Natural fat | 17.4 | 5.9 |
| Triheptanoin | | 35 |

TABLE 2
Blood acyl-carnitines levels indicate that triheptanoin is metabolised

| | | | fold | |
|---|---|---|---|---|
| Acyl-carnitine | Standard Diet | 35% Triheptanoin | increase | p |
| C3-Carnitine | 0.504 ± 0.065 | 1.622 ± 0.293 | 3.22 | 0.003 |
| C5-Carnitine | 0.078 ± 0.012 | 0.144 ± 0.012 | 1.85 | 0.002 |
| C7-Carnitine | 0.01 ± 0 | 0.022 ± 0.004 | 2.20 | 0.012 |
| C2-Carnitine | 20.80 ± 1.92 | 25.734 ± 3.53 | 1.24 | ns |
| C4-Carnitine | 0.252 ± 0.035 | 0.278 ± 0.052 | 1.10 | ns |
| Free Carnitine | 18.39 ± 1.29 | 20.77 ± 2.74 | 1.13 | ns |

TABLE 3
Few changes in brain citric acid cycle intermediates and metabolites in
SE and no SE mice fed triheptanoin

| | | | | | |
|---|---|---|---|---|---|
| | No SE mice | No SE mice | SE mice | SE mice | |
| Metabolites | Standard Diet | Triheptanoin | Standard Diet | Triheptanoin | ANOVA P |

CAC INTERMEDI

| | | | | | |
|---|---|---|---|---|---|
| citrate | 0.20 ± 0.01 | 0.21 ± 0.01 | 0.20 ± 0.02 | 0.2 ± 0.01 | 0.72 |
| succinate | 0.11 ± 0.01 | 0.11 ± 0.01 | 0.11 ± 0.01 | 0.10 ± 0.01 | 0.74 |
| Fumarate (%) | 100 ± 6.9 | 82.8 ± 3.8* | 86.1 ± 4.7 | 81.4 ± 2.9 | 0.028 |
| malate (%) | 100.0 ± 4.2 | 125.5 ± 7.7** | 77.1 ± 4.3*** | 80.1 ± 3.9 | <0.0001 |

CAC PRODUCTS

| | | | | | |
|---|---|---|---|---|---|
| glutamate | 23.64 ± 1.13 | 21.59 ± 0.92 | 21.01 ± 0.80 | 22.01 ± 1.65 | 0.52 |
| glutamine | 19.17 ± 5.23 | 12.91 ± 0.49 | 12.89 ± 0.40 | 13.15 ± 0.48 | 0.21 |
| GABA | 2.55 ± 0.11 | 2.36 ± 0.13 | 2.17 ± 0.07* | 2.16 ± 0.05 | 0.011 |
| aspartate (%) | 100.0 ± 13.9 | 104.0 ± 7.0 | 71.4 ± 6.3 | 76.6 ± 4.1 | 0.006 |

OTHERS

| | | | | | |
|---|---|---|---|---|---|
| BHB | 55.6 ± 4.5 | 108.9 ± 9.6*** | 63.9 ± 3.3 | 127.4 ± 15.7*** | <0.0001 |

TABLE 4
Anticonvulsant properties of triheptanoin compared to current
epilepsy treatments in acute and chronic epilepsy models

| | Triheptanoin | PHB | PHT | VPA | CBZ | LEV | KD |
|---|---|---|---|---|---|---|---|
| MEST (acute) | + | + | + | + | + | − | − |
| PTZ (acute) | − | + | − | + | − | − | − |
| 6 Hz (acute) | − | + | + | + | +/− | + | + |
| PTZ in PILO-SE | + | − | − | − | nd | nd | nd |
| rodents (chronic) | | | | | | | |
| Delay in corneal | + | nd | nd | nd | nd | + | nd |
| kindling (chronic) | | | | | | | |

### REFERENCES

Alvestad S, Hammer J, Eyjolfsson E, Qu H, Ottersen O P, Sonnewald U (2008) Limbic structures show altered glial-neuronal metabolism in the chronic phase of kainate induced epilepsy. Neurochem Res 33:257-266.
Barton M E, Klein B D, Wolf H H, White H S (2001) Pharmacological characterization of the 6 Hz psychomotor seizure model of partial epilepsy. Epilepsy Res 47:217-227.
Blanco M M, dos Santos J G, Jr., Perez-Mendes P, Kohek S R, Cavarsan C F, Hummel M, Albuquerque C, Mello L E (2009) Assessment of seizure susceptibility in pilocarpine epileptic and nonepileptic Wistar rats and of seizure reinduction with pentylenetetrazole and electroshock models. Epilepsia 50:824-831.
Borges K, Gearing M, McDermott D L, Smith A B, Almonte A G, Wainer B H, Dingledine R (2003) Neuronal and glial pathological changes during epileptogenesis in the mouse pilocarpine model. Exp Neurol 182:21-34.
Borges K, Gearing M, Rittling S R, Sorensen E S, Kotloski R, Denhardt D T, Dingledine R (2008) Characterization of osteopontin expression and function after status epilepticus. Epilepsia 49:1675-1685.
Borges K, McDermott D, Irier H, Smith Y, Dingledine R (2006) Degeneration and proliferation of astrocytes in the mouse dentate gyrus after pilocarpine-induced status epilepticus. Exp Neurol 201:416-427.
Borges K, McDermott D L, Dingledine R (2004) Reciprocal changes of CD44 and GAP-43 expression in the dentate gyrus inner molecular layer after status epilepticus in mice. Exp Neurol 188:1-10.
Bough K J, Wetherington J, Hassel B, Pare J F, Gawryluk J W, Greene J G, Shaw R, Smith Y, Geiger J D, Dingledine R J (2006) Mitochondrial biogenesis in the anticonvulsant mechanism of the ketogenic diet. Ann Neurol 60:223-235.
Brandt C, Heile A, Potschka H, Stoehr T, Loscher W (2006) Effects of the novel antiepileptic drug lacosamide on the development of amygdala kindling in rats. Epilepsia 47:1803-1809.
Curia G, Longo D, Biagini G, Jones R S, Avoli M (2008) The pilocarpine model of temporal lobe epilepsy. J Neurosci Methods 172:143-157.
Garriga-Canut M, Schoenike B, Qazi R, Bergendahl K, Daley T J, Pfender R M, Morrison J F, Ockuly J, Stafstrom C, Sutula T, Roopra A (2006)2-Deoxy-D-glucose reduces epilepsy progression by NRSF-CtBP-dependent metabolic regulation of chromatin structure. Nat Neurosci 9:1382-1387.
Giardina W J, Gasior M (2009) Acute seizure tests in epilepsy research:electroshock and chemical induced convulsions in the mouse. Current Protocols in Pharmacology 1:5.22.21-25.22.37.
Goldberg N D, Passonneau J V, Lowry O H (1966) Effects of changes in brain metabolism on the levels of citric acid cycle intermediates. J Biol Chem 241:3997-4003.
Hartman A L, Gasior M, Vining E P, Rogawski M A (2007) The neuropharmacology of the ketogenic diet. Pediatr Neurol 36:281-292.
Hartman A L, Lyle M, Rogawski M A, Gasior M (2008) Efficacy of the ketogenic diet in the 6-Hz seizure test. Epilepsia 49:334-339.
Kimball A W, Burnett Jr. W T, Doherty D G, (1957) Chemical protection against ionizing radiation I. Sampling methods for screening compounds in radiation protection studies in mice. Radiation Research 7:1-12.
Kinman R P, Kasumov T, Jobbins K A, Thomas K R, Adams J E, Brunengraber L N, Kutz G, Brewer W U, Roe C R, Brunengraber H (2006) Parenteral and enteral metabolism of anaplerotic triheptanoin in normal rats. Am J Physiol Endocrinol Metab 291:E860-866.
Klepper J, Leiendecker B (2007) GLUT1 deficiency syndrome—2007 update. Developmental medicine and child neurology 49:707-716.
Klitgaard H, Matagne A, Gobert J, Wulfert E (1998) Evidence for a unique profile of levetiracetam in rodent models of seizures and epilepsy. Eur J Pharmacol 353:191-206.
Kudin A P, Zsurka G, Elger C E, Kunz W S (2009) Mitochondrial involvement in temporal lobe epilepsy. Exp Neurol 218:326-332.
Lian X Y, Khan F A, Stringer J L (2007) Fructose-1,6-bisphosphate has anticonvulsant activity in models of acute seizures in adult rats. J Neurosci 27:12007-12011.
Loscher W, Honack D, Fassbender C P, Nolting B (1991) The role of technical, biological and pharmacological factors in the laboratory evaluation of anticonvulsant drugs. III. Pentylenetetrazole seizure models. Epilepsy Res 8:171-189.
Matagne A, Klitgaard H (1998) Validation of corneally kindled mice: a sensitive screening model for partial epilepsy in man. Epilepsy Res 31:59-71.
Matagne A, Margineanu D G, Kenda B, Michel P, Klitgaard H (2008) Anti-convulsive and anti-epileptic properties of brivaracetam (ucb 34714), a high-affinity ligand for the synaptic vesicle protein, SV2A. Br J Pharmacol 154:1662-1671.
Melo T M, Nehlig A, Sonnewald U (2005) Metabolism is normal in astrocytes in chronically epileptic rats: a (13)C NMR study of neuronal-glial interactions in a model of temporal lobe epilepsy. J Cereb Blood Flow Metab 25:1254-1264.
Neal E G, Chaffe H, Schwartz R H, Lawson M S, Edwards N, Fitzsimmons G, Whitney A, Cross J H (2008) The ketogenic diet for the treatment of childhood epilepsy: a randomised controlled trial. Lancet Neurol 7:500-506.
Nordstrom C H, Rehncrona S, Siesjo B K (1978) Effects of phenobarbital in cerebral ischemia. Part II: restitution of cerebral energy state, as well as of glycolytic metabolites, citric acid cycle intermediates and associated amino acids after pronounced incomplete ischemia. Stroke; a journal of cerebral circulation 9:335-343.
Potschka H, Loscher W (1999) Corneal kindling in mice: behavioral and pharmacological differences to conventional kindling. Epilepsy Res 37:109-120.
Puchowicz M A, Zechel J L, Valerio J, Emancipator D S, Xu K, Pundik S, LaManna J C, Lust W D (2008) Neuroprotection in diet-induced ketotic rat brain after focal ischemia. J Cereb Blood Flow Metab 28:1907-1916.
Racine R J (1972) Modification of seizure activity by electrical stimulation. II. Motor seizure. Electroencephalogr Clin Neurophysiol 32:281-294.
Rashed M S, Bucknall M P, Little D, Awad A, Jacob M, Alamoudi M, Alwattar M, Ozand P T (1997) Screening blood spots for inborn errors of metabolism by electrospray tandem mass spectrometry with a microplate batch process and a computer algorithm for automated flagging of abnormal profiles. Clin Chem 43:1129-1141.
Rowley N M, White H S (2010) Comparative anticonvulsant efficacy in the corneal kindled mouse model of partial epilepsy: Correlation with other seizure and epilepsy models. Epilepsy Res 92:163-169.
Samala R, Willis S, Borges K (2008) Anticonvulsant profile of a balanced ketogenic diet in acute mouse seizure models. Epilepsy Res 81:119-127.
Silver J M, Shin C, McNamara J O (1991) Antiepileptogenic effects of conventional anticonvulsants in the kindling model of epilespy. Ann Neurol 29:356-363.
Smith M, Wilcox K S, White H S (2007) Discovery of antiepileptic drugs. Neurotherapeutics 4:12-17.
Stafstrom C E, Ockuly J C, Murphree L, Valley M T, Roopra A, Sutula T P (2009) Anticonvulsant and antiepileptic actions of 2-deoxy-D-glucose in epilepsy models. Ann Neurol 65:435-447.
Turski W A, Cavalheiro E A, Bortolotto Z A, Mello L M, Schwarz M, Turski L (1984) Seizures produced by pilocarpine in mice: a behavioral, electroencephalographic and morphological analysis. Brain Res 321:237-253.
Turski W A, Cavalheiro E A, Schwarz M, Czuczwar S J, Kleinrok Z, Turski L (1983) Limbic seizures produced by pilocarpine in rats: behavioural, electroencephalographic and neuropathological study. Behav Brain Res 9:315-335.
Waldbaum S, Patel M (2010) Mitochondria, oxidative stress, and temporal lobe epilepsy. Epilepsy Res 88:23-45.
Wang X, Allen F J, Sayre C, Wan D, Minkler P E, Hoppel C L, Brunengraber H (2007) Anaplerosis from heptanoate—a propionyl-CoA precursor—in mouse brain. FASEB 21:541.512.
White H S (2003) Preclinical development of antiepileptic drugs: past, present, and future directions. Epilepsia 44 Suppl 7:2-8.
White H S, Woodhead J H, Wilcox K S, Stables J P, Kupferberg H J, Wolf H H (2002) Discovery and preclinical development of antiepileptic drugs. In: Antiepileptic drugs(Levy, R. H. et al., eds), pp 36-48 Philadelphia: Lippincott Williams & Wilkins.
Willis S, Samala R, Rosenberger T A, Borges K (2009) Eicosapentaenoic and docosahexaenoic acids are not anticonvulsant or neuroprotective in acute mouse seizure models. Epilepsia 50:138-142.

1. A method of treating an animal with epilepsy and/or other diseases or conditions that cause or result in seizures in an animal, wherein said method includes the step of administering a therapeutically effective amount of at least one precursor of propionyl-CoA to said animal in the absence of a ketogenic diet, to thereby treat said animal with epilepsy and/or other diseases or conditions that cause or result in seizures in said animal.

2. The method of claim 1, to treat an animal with epilepsy.

3. The method of treating an animal according to claim 1, wherein the at least one precursor of propionyl-CoA is selected from the group consisting of an uneven chain fatty acid, a triglyceride, a C5 ketone body, a phospholipid, a branched chain amino acid and combinations thereof.

4. The method of treating an animal according to claim 3, wherein the triglyceride is one or more compounds of Formula I: 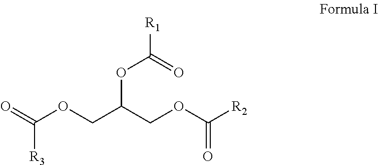
wherein
R₁, R₂ and R₃ are independently selected from alkyl, alkenyl or alkynyl.

5. The method of treating an animal according to claim 4, wherein R₁, R₂ and R₃ are independently selected from C₁ to C₂₀ alkyl, alkenyl or alkynyl.

6. The method of treating an animal according to claim 5, wherein R₁, R₂ and R₃ are the same and are selected from the group consisting of C₅, C₆, C₇, C₈ and C₉ alkyl.

7. The method of treating an animal according to claim 6, wherein the compound of Formula I is selected from tripentanoin, triheptanoin and trinonanoin.

8. The method of claim 7, wherein the compound is triheptanoin.

9. The method of treating an animal according to claim 1, wherein the at least one precursor of propionyl-CoA is provided to the animal in an amount comprising at least about 5% of the dietary caloric intake for the animal.

10. The method of treating an animal according to claim 9, wherein the at least one precursor of propionyl-CoA is provided to the animal in an amount comprising at least about 20% of the dietary caloric intake for the animal.

11. The method of treating an animal according to claim 10, at least one precursor of propionyl-CoA is provided to the animal in an amount comprising at least about 30% of the dietary caloric intake for the animal.

12. The method of treating an animal according to claim 11, at least one precursor of propionyl-CoA is provided to the animal in an amount comprising at least about 35% of the dietary caloric intake for the animal.

13. The method of treating an animal according to claim 1, wherein the animal is an adult animal.

14. The method of treating an animal according to claim 1, the animal is a mammal.

15. The method of treating an animal according to claim 14, the mammal is a human.

16. The method of treating an animal according to claim 1, wherein the step of administering a therapeutically effective amount of at least one precursor of propionyl-CoA is oral administration.

17. A pharmaceutical composition for use in the treatment of an animal with epilepsy and/or other diseases or conditions that cause or result in seizures in an animal in the absence of a ketogenic diet, said pharmaceutical composition comprising a therapeutically effective amount of at least one precursor of propionyl-CoA together with a pharmaceutically acceptable carrier, diluent or excipient.

18. The pharmaceutical composition of claim 17, wherein the at least one precursor of propionyl-CoA is selected from the group consisting of an uneven chain fatty acid, a triglyceride, a C5 ketone body, a phospholipid, a branched chain amino acid and combinations thereof.

19. The pharmaceutical composition according to claim 17, wherein the at least one precursor of propionyl-CoA is a triglyceride.

20. The pharmaceutical composition of claim 17, which is a dietary formulation or nutritional supplement suitable for oral administration.
